(19) United States
(12) Patent Application Publication
(10) Pub. No.: US 2026/0087617 A1
(43) Pub. Date: Mar. 26, 2026

(54) SYSTEMS AND METHODS FOR MEASURING FLOW PROPAGATION VELOCITY FROM MULTI-DIMENSIONAL CARDIAC IMAGING

(71) Applicant: Purdue Research Foundation, West Lafayette, IN (US)

(72) Inventors: Pavlos Vlachos, West Lafayette, IN (US); Jiacheng Zhang, Brookfield, WI (US)

(21) Appl. No.: 19/109,877

(22) PCT Filed: Sep. 7, 2023
(86) PCT No.: PCT/US2023/032134

§ 371 Date: Mar. 7, 2025

Related U.S. Application Data
(60) Provisional application No. 63/374,926, filed on Sep. 8, 2022.

(51) Int. Cl. G06T 7/00; A61B 5/026

(52) U.S. Cl. CPC G06T 7/0012; A61B 5/0263; A61B 2576/023; G06T 2207/10088; G06T 2207/30048; G06T 2207/30104

(57) ABSTRACT

The invention generally provides systems and methods for measuring flow propagation velocity from multi-dimensional cardiac imaging. In certain aspects, the invention provides systems and methods for measuring propagation velocity from multi-dimensional cardiac imaging that involve receiving cardiac imaging data; estimating local and instantaneous flow propagation velocity (Vₚᵣₒₚ) from the cardiac imaging data; and employing the local and instantaneous flow propagation velocity to evaluate cardiac flow propagation.

### RELATED APPLICATION

The present application claims the benefit of and priority to U.S. provisional patent application Ser. No. 63/374,926, filed Sep. 8, 2022, the content of which is incorporated by reference herein in its entirety.

### GOVERNMENT SUPPORT

This invention was made with government support under NS106696 and HL115267 awarded by the National Institutes of Health. The government has certain rights in the invention.

### FIELD OF THE INVENTION

The invention generally provides systems and methods for measuring flow propagation velocity from multi-dimensional cardiac imaging.

### BACKGROUND

A large portion of the patients with heart failure have a preserved left ventricular (LV) ejection fraction (HFpEF) and suffer from left ventricular diastolic dysfunction (LVDD).

Impaired relaxation and reduced ventricular compliance are the hallmarks of LVDD, and the propagation velocity of the inflow jet during the LV early diastole has been proposed as a marker of the diastolic function. The normal LV relaxation creates a pressure drop from mitral orifice towards apex and enhances the vortex ring at the mitral valve tips, aiding the early diastolic filling. The propagation velocity of normal filling can exceed the speed of the blood cell and resembles the motion of an entire column of blood with short delay between the occurrence of peak velocity at mitral tip and apical region. With left ventricular diastolic dysfunction (LVDD), the LV relaxation is impaired, reducing the pressure force towards the apex and subsequently slowing the flow propagation.

The conventional propagation velocity (Vp) is determined from the spatiotemporal velocity map provided by Color M-mode (CMM) echocardiography as the slope of the iso-velocity contour corresponding to the front of the inflow wave. Vp can also be quantified based on the time difference between the occurrences of peak velocity in the apical region and at the mitral tip. These methods assume a constant Vp during early diastole, while the inflow wave front shows a curvilinear feature, suggesting that Vp is spatially and temporally varying, and therefore multiple Vp values can be extracted from a single image based on different iso-velocity contours. As a consequence, large differences were found between the Vp obtained using different methods due to a lack of consensus on the definition of Vp. Recent developments consider the spatiotemporal variation of Vp and improved the classification ability. However, the existing Vp measurement methods are only applicable to CMM echocardiography which provides one-dimensional measurement, and the accuracy of the velocity and Vp is affected by the angle between M-mode cursor and flow.

### SUMMARY

The invention provides new systems and methods to evaluate left ventricular (LV) diastolic flow propagation from cardiac flow imaging. The proposed approach estimates local and instantaneous flow propagation velocity (Vₚᵣₒₚ) by fitting a first order wave equation to velocity gradients with weighted least-squares. The proposed approach was validated using the synthetic vortex ring flow data created with one, two, or three spatial dimensions and different noise levels. The validation results indicate that the error in the Vₚᵣₒₚ estimated from two-dimensional and three-dimensional data is about 50% lower than the Vₚᵣₒₚ error from one-dimensional data. The approach was applied to the velocity fields acquired with two-dimensional phase-contrast magnetic resonance imaging (pc-MRI) and 4D flow MRI and provided the spatial distribution and the temporal evolution of Vₚᵣₒₚ during the LV diastole. During early diastole of a normal left ventricle, the timing of peak flow propagation coincides with the peak intraventricular pressure difference instead of the peak mitral inflow, suggesting that the pressure gradient created by the normal LV relaxation has a greater effect on the flow propagation towards apex. The flow propagation towards the apex is mainly located at the front of the inflow jet, and the vortex ring formed near the mitral valve tips aid the LV filling propagation. The proposed approach provides a more comprehensive investigation and potentially improves the evaluation of LV diastolic function.

In certain aspects, the invention provides methods for measuring propagation velocity from multi-dimensional cardiac imaging that involve receiving cardiac imaging data; estimating local and instantaneous flow propagation velocity (Vₚᵣₒₚ) from the cardiac imaging data; and employing the local and instantaneous flow propagation velocity to evaluate cardiac flow propagation.

In other aspects, the invention provides systems for measuring propagation velocity from multi-dimensional cardiac imaging. The systems of the invention include a processor configured to: receive cardiac imaging data; estimate local and instantaneous flow propagation velocity (Vₚᵣₒₚ) from the cardiac imaging data; and employ the local and instantaneous flow propagation velocity to evaluate cardiac flow propagation.

In certain embodiments of the systems and methods of the invention, the cardiac imaging data is 4D magnetic resonance imaging (MRI) data. In certain embodiments of the systems and methods of the invention, the local and instantaneous flow propagation velocity (Vₚᵣₒₚ) is determined by fitting a first order wave equation to velocity gradients with weighted least-squares. In certain embodiments of the systems and methods of the invention, the Vₚᵣₒₚ is estimated from velocity gradients numerically calculated from the velocity fields using second order central (SOC) difference scheme.

In certain embodiments of the systems and methods of the invention, for each timeframe, the Vₚᵣₒₚ at each spatial point is determined by the weighted least-squares (WLS) fitting of a wave propagation equation as:

$${V}_{prop} = arg min ( {\sum }_{i}^{n} {w}_{i}^{2} {\vert \frac{\partial \vec{u}}{\partial t} + {V}_{prop} \cdot ∇ \vec{u} \vert}_{i}^{2} )$$

where n is the total number of data points within the field, and wᵢ is the weight for the i-th data point. In certain embodiments of the systems and methods of the invention, the i-th data point, is generated based on its spatial distance |Δ [symbol] | from the point of interest as:

$${w}_{i} = \{ \begin{matrix}exp ( - \frac{{\vert Δ \vec{x} \vert}^{2}}{{L}_{0}} & if \vert Δ \vec{x} \vert < {L}_{0} \\ 0 & else\end{matrix}$$

where L₀=0.5 cm is the length scale, yielding a kernel width of 1 cm which corresponds approximately to the radius of the mitral valve. In certain embodiments of the systems and methods of the invention, weight decreases with increase of the distance |Δ [symbol] |, and only data within L₀ is employed for the fitting.

In certain embodiments of the systems and methods of the invention, the Vₚᵣₒₚ that is dependent on a local flow structure. In certain embodiments of the systems and methods of the invention, the approach further comprising quantifying relative strength of the propagation in a manner in which the Vₚᵣₒₚ component along a direction from mitral orifice towards an apex is extracted and spatially integrated in the LV. In certain embodiments of the systems and methods of the invention, an integral at each timeframe is normalized by an average of all the timeframes during diastole and is named as propagation intensity (Iₚᵣₒₚ).

### BRIEF DESCRIPTION OF THE DRAWINGS

FIG. 1 panel A demonstrates the prior art common approach to measure Vp from the spatiotemporal velocity map. The black contour represents the velocity at 50% of the peak diastolic transmitral velocity. The Vp is estimated as the slope of green dashed line which is the linear approximation of the E-wave front based on the iso-velocity contour. Panel B demonstrates the relationship between the Vp and the velocity values at different spatiotemporal points within the region of the velocity map indicated using the black dashed box shown in Panel A.

FIG. 2 panel A shows the three-dimensional velocity field of the vortex ring flow from the middle timeframe. The color of the arrows corresponds to the local velocity magnitude. Panel B shows the two-dimensional velocity field of the vortex ring flow on the center x-z plane from the middle timeframe. The background color suggests the local velocity magnitude. Panel C shows the spatiotemporal velocity map sampled along the z-axis.

FIG. 3 shows the normalized absolute error in the Vₚᵣₒₚ calculated using the proposed method from the 1D, 2D, and 3D data of the vortex ring flow as a function of the velocity noise level. The lines suggest the median absolute error, and the shaded region indicates the interquartile range of the error distribution.

FIG. 4 panel A shows the waveforms of the mitral inflow velocity, the intraventricular pressure difference (IVPD), and the propagation intensity (Iₚᵣₒₚ) during the LV diastole of the normal filling patient imaged with two-dimensional pc-MRI. Panel B shows the fields of the blood flow velocity, the Vp, and the relative pressure from the timeframes indicated using the dotted lines in the waveform plots in Panel A.

FIG. 5 panel A shows the waveforms of the mitral inflow velocity, the intraventricular pressure difference (IVPD), and the propagation intensity (Iₚᵣₒₚ) during the LV diastole of the LVDD patients imaged with two-dimensional pc-MRI. The left column shows the HCM patient, and the right column corresponds the DCM patient. Panel B shows the fields of the blood flow velocity, the Vp, and the relative pressure from the timeframes indicated using the dotted lines in the waveform plots in Panel A.

FIG. 6 panel A shows the waveforms of the mitral inflow velocity, the intraventricular pressure difference (IVPD), and the propagation intensity (Iₚᵣₒₚ) during the LV diastole determined from the 4D flow MRI data of a normal filling subject. Panel B shows the fields of the blood flow velocity, the Vₚᵣₒₚ, and the relative pressure on the four-chamber view from the timeframes indicated using the dotted lines in the waveform plots in Panel A.

FIG. 7 is a high-level diagram showing the components of an exemplary data-processing system.

### DETAILED DESCRIPTION

The invention introduces a new approach for determining the propagation velocity from cardiac flow data and to resolve the spatiotemporal variations of Vp. This enables investigation of the correlation between Vp and the complex flow structures observed in the LV. The approach was validated using synthetic flow data of a self-induced vortex ring. The approach was demonstrated using in vivo data acquired using two-dimensional phase-contrast magnetic resonance imaging (pc-MRI) and 4D flow MRI.

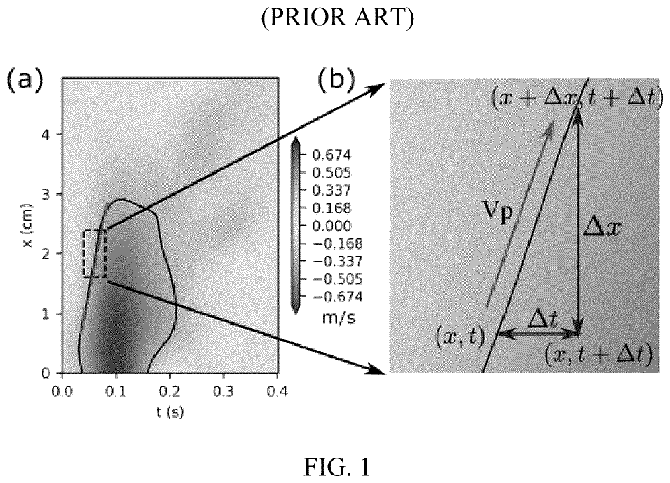

FIG. 1 panel A illustrates the conventional approach (prior art) to measure Vp from the spatiotemporal velocity map as the slope of a linear approximation of the iso-velocity contour. This approach estimates the flow propagation by tracking the spatiotemporal occurrence of the contour-level velocity, e.g., from (x, t) to (x+Δx, t+Δt) with Vp=Δx/Δt as demonstrated in FIG. 1 panel B. Alternatively, the propagation can be inferred from the following relationship between the velocity values and gradients at different spatiotemporal points as:

$$u ( x , t + Δ t ) = u ( x + Δ x , t + Δ t ) - \frac{\partial u}{\partial x} Δ x = u ( x , t ) + \frac{\partial u}{\partial t} Δ t , \qquad ( 1 )$$

where ∂u/∂t and ∂u/∂x are the temporal (t) and spatial (x) velocity gradients, respectively. Since both (x, t) and (x+Δt, t+Δt) are on the iso-velocity contour line, u(x, t)=u(x+Δt, t+Δt), and the following formulation can be derived from equation (1) as:

$$\frac{\partial u}{\partial t} + Vp \frac{\partial u}{\partial x} = 0. \qquad ( 2 )$$

Equation (2) is the first order wave equation governing the propagation of a waveform denoted by u(x, t). With multi-dimensional and multi-component velocity data [symbol] ( [symbol] , t), Equation (2) can be modified as:

$$\frac{\partial \vec{u}}{\partial t} + \vec{Vp} \cdot ∇ \vec{u} = 0 , \qquad ( 3 )$$

where ∇ represents the spatial gradient operator, and [symbol] the vector consisting of the propagation velocity along all spatial dimensions. Equations (2) and (3) suggest that Vp can be estimated from the velocity gradients. We use Vₚᵣₒₚ to denote the propagation velocity estimated based on the first order wave equation herein, which is a scalar if estimated from one-dimensional data and a vector if estimated from multi-dimensional data.

The Vₚᵣₒₚ was estimated from the velocity gradients numerically calculated from the velocity fields using second order central (SOC) difference scheme. For each timeframe, the Vₚᵣₒₚ at each spatial point was determined by the weighted least-squares (WLS) fitting of the wave propagation equation (3) as:

$${V}_{prop} = arg min ( {\sum }_{i}^{n} {w}_{i}^{2} {\vert \frac{\partial \vec{u}}{\partial t} + {V}_{prop} \cdot ∇ \vec{u} \vert}_{i}^{2} ) , \qquad ( 4 )$$

where n is the total number of data points within the field, and wᵢ is the weight for the i-th data point which was generated based on its spatial distance |Δ [symbol] | from the point of interest as:

$${w}_{i} = \{ \begin{matrix}exp ( - \frac{{\vert Δ \vec{x} \vert}^{2}}{{L}_{0}} & if \vert Δ \vec{x} \vert < {L}_{0} \\ 0 & else\end{matrix} , \qquad ( 5 )$$

where L₀=0.5 cm is the length scale, yielding a kernel width of 1 cm which corresponds approximately to the radius of the mitral valve. The weight decreases with the increase of the distance |Δ [symbol] |, an n data within L₀ is employed for the fitting. The proposed WLS optimization will yield Vₚᵣₒₚ that is dependent on the local flow structure and ensures the robustness of the fitting.

To quantify the relative strength of the propagation, the Vₚᵣₒₚ component along the direction from mitral orifice towards the apex is extracted and spatially integrated in the LV. The integral at each timeframe is normalized by the average of all the timeframes during diastole and is named as the propagation intensity (Iₚᵣₒₚ).

Synthetic flow fields of a self-induced Lamb-Oseen vortex ring were created to assess the accuracy of the proposed Vₚᵣₒₚ calculation method. The radius of the circular vortex ring (r₀) is 2 cm, and the angular velocity relative to the ring's circular axis can be expressed as:

$${u}_{θ} = {u}_{max} ( 1 + \frac{1}{2 α} ) \frac{{r}_{max}}{r} ( 1 - {e}^{- α \frac{{r}^{2}}{{r}_{max}^{2}}} ) , \qquad ( 7 )$$

where uₘₐₓ=0.5 m/s is the maximum angular velocity, rₘₐₓ=\sqrt{α}×r_{c} is the distance from the vortex core where the maximum angular velocity is reached, r_{c}=0.5 cm is the vortex core radius, and the constant α=1.25643. The self-induction velocity (u₀) of the vortex ring is along the z-axis and can be determined from:

$${u}_{0} = {u}_{max} ( 1 + \frac{1}{2 α} ) \frac{{r}_{max}}{2 {r}_{0}} ( 1 - {e}^{- α \frac{{r}_{0}^{2}}{{r}_{max}^{2}}} ) , \qquad ( 8 )$$

which is considered as the “ground truth” propagation velocity of the vortex ring flow. Three-dimensional (3D) velocity fields were created on a Cartesian grid with a spatial resolution of 2 mm in a domain spanning from −2r₀ to 2r₀ along each spatial dimension. A total of 41 timeframes were uniformly sampled during the time when the vortex ring propagated from z=−r₀ to z=r₀, yielding a sampling rate at 98 Hz. In addition to the 3D data, two-dimensional (2D) and one-dimensional (1D) datasets were extracted at the center z-x plane and along the z-axis, respectively. To test the robustness of the proposed method against measurement noise, normally distributed random noise was added to the velocity data with the standard deviation varying from 0% to 20% of the maximum velocity magnitude along the z-axis.

Two-dimensional pc-MRI measurements were acquired from three patients, one with normal filling, one with LVDD and hypertrophic cardiomyopathy (HCM), and one with LVDD and dilated cardiomyopathy (DCM), in accordance with the pre-established Institutional Review Board guidelines. The scans were performed at the Wake Forest University Baptist Medical Center in Winston-Salem, NC on an Avanto 1.5T scanner from Siemens Medical Solutions. Velocity encoding (VENC) was 100-130 cm/s, with a repetition time (TR) of 20 ms and an echo time (TE) of 3.3 ms. Flip angle was 20°, and the spatial resolution was 1.25 mm/pixel in-plane with a 5-mm slice thickness. Retrospective ECG gating was used for the acquisition with 40 or 45 reconstructed phases depending on patient heart rate. The pc-MRI images were segmented based on a separate high signal-to-noise ratio imaging scan acquired over the same field of view, and the time-dependent LV boundaries were created for the pc-MRI fields. These data have been used in previous studies.

4D flow MRI data were acquired for three subjects with normal LV diastole at the Children's National Hospital in an Institutional Review Board-approved retrospective study. A Siemens 1.5-T scanner was used for acquiring the CMR data, with the field of view (FOV) of 280-480×140-230 mm and a matrix of 160×77. The TE was 2.19 ms, and the TR was 37.9-59.4 ms. The flip angle was 15°, and the VENC was 2-2.5 m/s. The slice size was 1.8 mm or 2.75 mm, and the pixel size was 1.75 or 2.735 mm, depending on the patient size. The number of reconstructed phases was 20-30 of a cardiac cycle. The time-dependent LV boundaries for the 4D flow data were created based on the long-axis and short-axis cine images acquired for the same subjects.

The following preprocessing procedure was performed on the velocity fields of the synthetic data and the in vivo cardiac flow prior to the Vₚᵣₒₚ estimation. The spurious velocity measurements were detected using the universal outlier detection (UOD) method based on the local variation of velocity in the neighborhood containing 3 pixels along each spatial dimension, and the outlier measurements were replaced with the median of the neighborhood. To ensure the smoothness of the velocity field, the velocity profile along each dimension was reconstructed as an ensemble of radial basis functions (RBFs):

$$u ( x ) = {\sum }_{j = 1}^{N} {s}_{j} {T}_{j} ( {r}_{j} ) , \qquad ( 9 )$$

$$with T ( r ) = {r}^{4} ln r , \qquad ( 10 )$$

where N is the total number of RBFs, sⱼ is the amplitude of the j-th RBF Tⱼ, and rⱼ=|x−xⱼ| is the distance to the j-th RBF centered at xⱼ. The 4-th order thin-plate spline is employed for the RBF as expressed in equation (10). The RBFs were distributed uniformly along each dimension with 5 mm separation. The RBF amplitudes were determined as:

$${s}_{j} = arg min ( {\vert {\sum }_{j = 1}^{N} {s}_{j} {T}_{j} ( {r}_{j} ) - u \vert}^{2} ) , \qquad ( 11 )$$

which minimizes the least-squares residual between the original velocity profile with the RBF representation to ensure the fidelity of the reconstruction.

Instantaneous pressure fields were estimated from the LV velocity fields using the measurement-error based WLS method. The pressure gradients (p_{grad}) were calculated using the Navier-Stokes momentum equation, which were then spatially integrated to obtain the pressure field (p_{WLS}) as:

$${p}_{WLS} = arg min ( {\vert W ( ∇ p - {p}_{grad} ) \vert}^{2} ) , \qquad ( 12 )$$

where W is the weight matrix generated based on the velocity error predicted from the spurious divergence of the velocity field. A 0 Pa reference pressure was assigned at the mitral orifice such that the estimated pressure is relative to the mitral orifice. The pressure difference between the mitral orifice and the apical region is defined as the intraventricular pressure difference (IVPD). A positive IVPD means that the pressure at the mitral orifice is higher than the pressure in the apical region.

The vortical structures were identified from the LV velocity fields based on the local swirling strength denoted as λ_{ci} which is quantified as the imaginary part of the complex eigenvalues of the velocity gradient tensor. Vortices were identified as the connected regions where the absolute value of λ_{ci} is above 4% of the maximum value measured in the LV over the diastole.

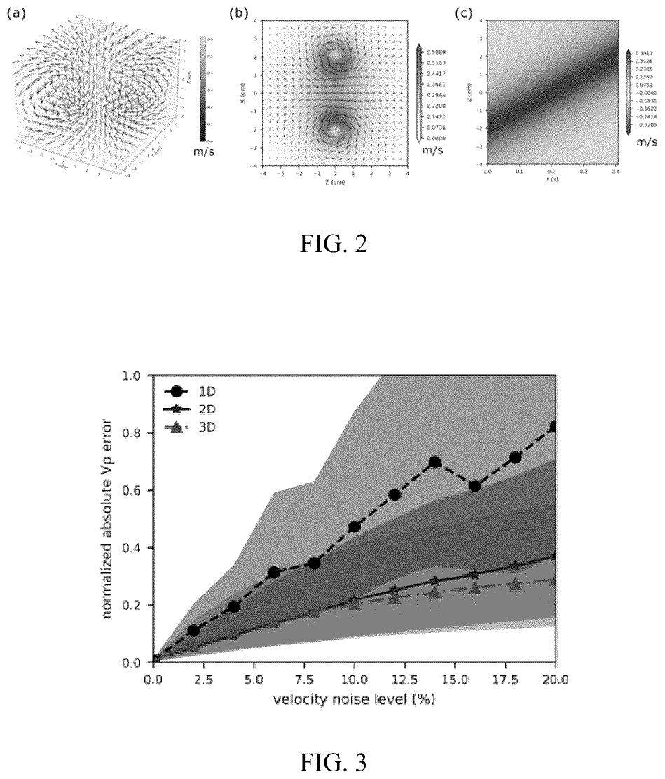

FIG. 2 panels A-B show the velocity field in the 3D volume and on the center x-z plane, respectively, from the middle timeframe when the vortex ring center is located at z=0 mm. FIG. 2 (c) presents the spatiotemporal velocity map of the 1D data sampled along the z-axis. The errors in the estimated Vₚᵣₒₚ were assessed as the differences from the vortex ring's self-induction velocity u₀. For each dataset, the quartiles of the absolute Vₚᵣₒₚ errors were determined in a moving region defined as |z-zₒ|<r₀, where zₒ is the z-location of the vortex ring center which propagates from −r₀ to r₀ during the sampled time. The quartiles are normalized by u₀ and shown in FIG. 3 as a function of the velocity noise level. The normalized median absolute error in the Vₚᵣₒₚ estimated from the 1D data increases from 0.007 to 0.82 as the velocity noise level increases from 0% to 20%, while the normalized median absolute Vp error increases from 0.008 to 0.37 and from 0.004 to 0.29 for the estimations from 2D and 3D datasets, respectively.

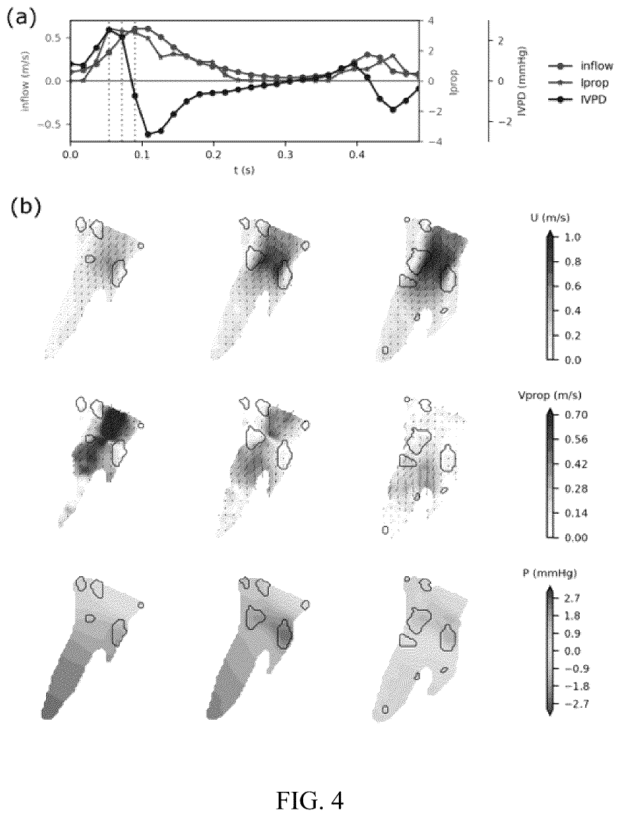

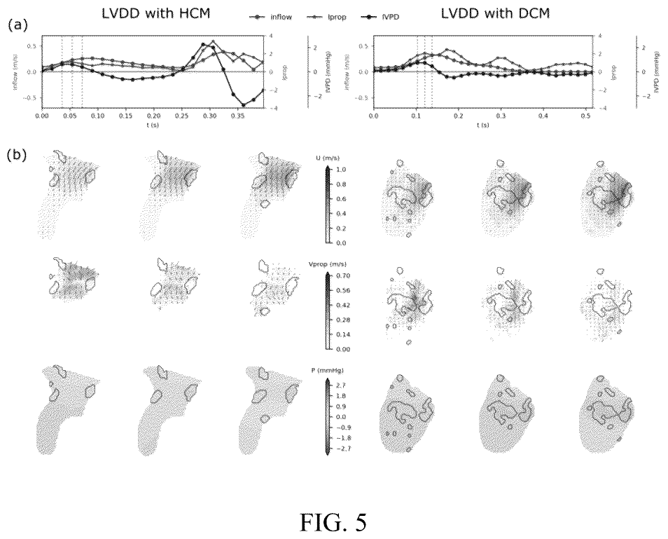

FIG. 4 panel A shows the waveforms of the mitral inflow, the IVPD, and the propagation intensity (Iₚᵣₒₚ) during LV diastole for the normal filling patient. At the beginning of the normal LV filling, the IVPD and Iₚᵣₒₚ increases as the inflow velocity increases. The peak Iₚᵣₒₚ coincides with the peak IVPD at around 0.05 s after the start of the LV diastole. The IVPD quickly drops to negative when the peak inflow velocity is reached, suggesting that the pressure in the apical region becomes higher than the mitral orifice pressure. The secondary peaks of the Iₚᵣₒₚ and IVPD can be observed during the atrial filling around 0.4 s. For LVDD patients shown in FIG. 5 panel A, the peaks of the mitral inflow velocity and IVPD during early diastole are lower than the normal filling. The peaks of Ip and IVPD are higher during the atrial filling at around 0.3 s than the peaks during early diastole for the HCM patient, while the DCM patient shows no prominent peaks of IVPD or Iₚᵣₒₚ during the entire diastole.

The fields of flow velocity, Vₚᵣₒₚ, and relative pressure in the LV at three consecutive timeframes during early diastole are presented in FIG. 4 panel B and FIG. 5 panel B for the normal filling and LVDD patients, respectively. The plotted timeframes are indicated using the vertical dotted lines in the corresponding waveform plots. The black contours in the fields identify the borders of the vortex structures. For the normal filling, a vortex ring is formed near the mitral valve tips around the inflow jet, and strong flow propagation towards apex can be observed downstream of the vortex ring. The pressure decreases from the mitral orifice to the apex at the first timeframe, while the pressure in the apical region rises and becomes higher than the pressure around the mitral orifice at the third timeframe. From the LVDD patients, both the inflow jet and the flow propagation are weaker than the normal filling. The flow propagation of the LVDD patients also shows a shorter penetration than the normal filling, as the filling Vₚᵣₒₚ is only found near the vortex ring from the LVDD patients, while the filling Vₚᵣₒₚ is still significant further downstream of the vortex ring into the apical region of the normal LV. The Vp downstream of the vortex ring is more aligned towards the apex in the normal LV, while the Vₚᵣₒₚ 's direction quickly diverges after passing the vortex ring in the LVDD patients. The pressure has a more uniform distribution in the LV of the LVDD patients than in the normal LV.

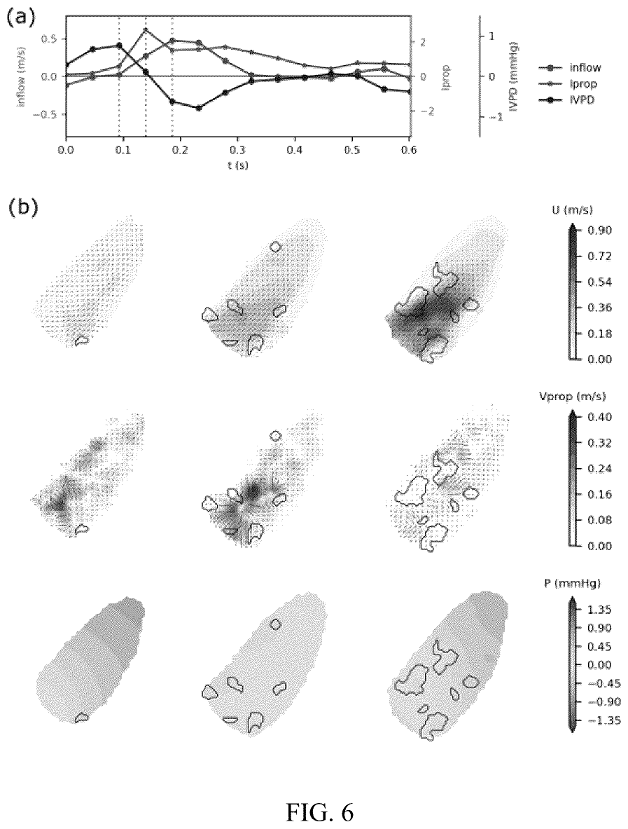

The waveforms of the mitral inflow, the IVPD, and Iₚᵣₒₚ determined from the 4D flow data of a normal subject are presented in FIG. 6 panel A. Positive IVPD is found at the beginning of the diastole as the mitral inflow increases and drops to negative when the peak inflow is reached. The peak of Iₚᵣₒₚ during early diastole is found between the peak IVPD and peak mitral inflow. Three timeframes during early diastole with increasing mitral inflow are selected as indicated by the vertical dotted lines. FIG. 6 panel B shows the in-plane velocity, Vₚᵣₒₚ and relative pressure fields during the selected timeframes on the four-chamber view. Like the results from the normal filling patient shown in FIG. 4 panels A-B, a vortex ring forms with the mitral inflow, and the relative pressure in the apical region rises as the inflow jet reaches the apex. As shown in the middle frame in FIG. 5 panel B, the region with significant filling Vₚᵣₒₚ towards apex is located downstream of the vortex ring. The waveforms and the fields of the other two subjects are provided in the supplementary material.

This study introduces a method to measure the LV filling propagation velocity from multi-dimensional cardiac flow imaging. The proposed method estimates the Vₚᵣₒₚ at each spatiotemporal point by fitting the first order wave equation to the velocity gradients in the neighborhood. The method's performance was evaluated with synthetic vortex ring flow data, and the error analysis results suggested that more accurate Vₚᵣₒₚ, can be obtained from multi-dimensional data (2D and 3D) than from 1D data. Compared to the result from 1D data with 20% noise, the median absolute Vₚᵣₒₚ, error was 55% and 65% lower from the 2D data and 3D data with the same noise level, respectively. Determining Vₚᵣₒₚ, from multi-dimensional data also avoids the limitation of the one-dimensional CMM that the measurement accuracy is affected by the angle between the M-mode cursor and the flow. The Vₚᵣₒₚ estimated from multi-dimensional data is also directional as shown in the Vₚᵣₒₚ fields from FIG. 4 panels A-B, FIG. 5 panels A-B, and FIG. 6 panels A-B.

The proposed method provides the spatial distribution and the temporal evolution of Vₚᵣₒₚ, which helps in understanding the mechanism of the LV filling propagation and its relationship with the pressure gradient and the vortical structures. For the normal filling shown in FIG. 4, the peak flow propagation in terms of Iₚᵣₒₚ occurs around the time when the maximum IVPD is reached with relatively low mitral inflow. At the later timeframes with the peak mitral inflow, the inflow jet reaches the apical region and increases the apical pressure. The pressure gradient no longer aids LV filling, and the Vₚᵣₒₚ becomes lower than the previous timeframes despite the stronger convection caused by the blood flow towards apex. This suggests that the pressure gradient created by the LV relaxation has a stronger effect on the flow propagation during early diastole than the local convection, which is consistent with the previous findings based on CMM echocardiography. For the LVDD patients, the timing of the peak Iₚᵣₒₚ also coincides with the peak IVPD during the early diastole as shown in FIG. 4 panels A-B, although the mitral inflow, IVPD, and Vₚᵣₒₚ are significantly lower than the values from the normal filling patient. Additionally, the Vₚᵣₒₚ is correlated with the vortex ring formed near the mitral valve tips during the early diastole. As shown in FIG. 4 panels A-B, FIG. 5 panels A-B, and FIG. 6 panels A-B, the flow propagation towards apex is mainly found at the front of the inflow jet downstream of the vortex ring. For the normal filling, the vortex ring creates a virtual channel, allowing the inflow jet to propagate into the LV without spreading. For the LVDD patients, the vortex ring at the mitral valve tips is smaller in size and closer to the base of the ventricle, leading to the shorter penetration of the flow propagation which quickly diverges after passing the vortex ring as shown in FIG. 5 panels A-B. The proposed method benefits the physical and physiological investigation of flow propagation by resolving its spatial and temporal distributions, which are not captured by the conventional methods.

There could be instances in which the Vₚᵣₒₚ is estimated from the velocity gradients whose accuracy is sensitive to the noise in the velocity data. To address such instances, we performed UOD followed by the RBF reconstruction to enhance the smoothness and the fidelity of the velocity data and therefore to ensure the reliability of the velocity gradient evaluation. Moreover, the Vₚᵣₒₚ measurement requires time-resolved velocity data. The maximum resolvable Vₚᵣₒₚ from the proposed method can be approximated as 0.5Lₚ/Δt, where Lₚ is the flow propagation distance, and Δt is the time difference between acquired phases. The factor 0.5 is due to the SOC scheme which estimates the temporal derivative from two timeframes separated by 2Δt. With a typical Lₚ of 4 cm, the minimum sampling rate required to resolve a common normal filling Vₚᵣₒₚ at 1 m/s is 50 Hz (Δt=25 ms), which can be difficult to achieve for some imaging modality such as 4D flow MRI. In the present study, the maximum normal filling Vₚᵣₒₚ obtained from the 4D flow MRI is around 0.4 m/s, which is lower than the maximum Vₚᵣₒₚ determined from the two-dimensional pc-MRI data at about 0.8 m/s. This may be caused by the difference in the temporal resolutions as the 4D flow data was acquired with a Δt of 28-46 ms, while the two-dimensional pc-MRI has a Δt of 18 ms.

Overall, this study introduces a novel flow propagation velocity measurement method for multi-dimensional cardiac flow imaging. The method estimates the Vₚᵣₒₚ by fitting the first order wave equation to the velocity gradients and can resolve the spatiotemporal variation of Vₚᵣₒₚ. The error analysis with synthetic vortex ring flow suggests that measuring Vₚᵣₒₚ from multi-dimensional data is more robust than from 1D data. The method was applied to the multi-dimensional CMR data and demonstrated the Vₚᵣₒₚ 's distribution in the LV and the evolution during the diastole. The results also reveal that the flow propagation during the early diastole is mainly driven by the pressure gradient, and the vortex ring formation near the mitral valve tips can aid the flow propagation.

### System Architecture

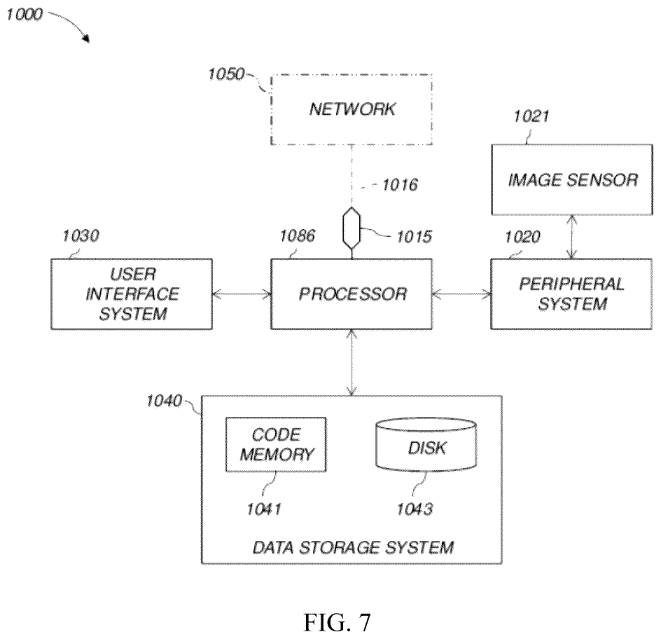

FIG. 7 is a high-level diagram showing the components of an exemplary data-processing system 1000 for analyzing data and performing other analyses described herein, and related components. The system includes a processor 1086, a peripheral system 1020, a user interface system 1030, and a data storage system 1040. The peripheral system 1020, the user interface system 1030 and the data storage system 1040 are communicatively connected to the processor 1086. Processor 1086 can be communicatively connected to network 1050 (shown in phantom), e.g., the Internet or a leased line, as discussed below. The data described above may be obtained using detector 1021 and/or displayed using display units (included in user interface system 1030) which can each include one or more of systems 1086, 1020, 1030, 1040, and can each connect to one or more network(s) 1050. Processor 1086, and other processing devices described herein, can each include one or more microprocessors, microcontrollers, field-programmable gate arrays (FPGAs), application-specific integrated circuits (ASICs), programmable logic devices (PLDs), programmable logic arrays (PLAs), programmable array logic devices (PALs), or digital signal processors (DSPs).

Processor 1086 which in one embodiment may be capable of real-time calculations (and in an alternative embodiment configured to perform calculations on a non-real-time basis and store the results of calculations for use later) can implement processes of various aspects described herein. Processor 1086 can be or include one or more device(s) for automatically operating on data, e.g., a central processing unit (CPU), microcontroller (MCU), desktop computer, laptop computer, mainframe computer, personal digital assistant, digital camera, cellular phone, smartphone, or any other device for processing data, managing data, or handling data, whether implemented with electrical, magnetic, optical, biological components, or otherwise. The phrase “communicatively connected” includes any type of connection, wired or wireless, for communicating data between devices or processors. These devices or processors can be located in physical proximity or not. For example, subsystems such as peripheral system 1020, user interface system 1030, and data storage system 1040 are shown separately from the data processing system 1086 but can be stored completely or partially within the data processing system 1086.

The peripheral system 1020 can include one or more devices configured to provide digital content records to the processor 1086. For example, the peripheral system 1020 can include medical devices (such as medical imaging devices), digital still cameras, digital video cameras, cellular phones, or other data processors. The processor 1086, upon receipt of digital content records from a device in the peripheral system 1020, can store such digital content records in the data storage system 1040.

The user interface system 1030 can include a mouse, a keyboard, another computer (e.g., a tablet) connected, e.g., via a network or a null-modem cable, or any device or combination of devices from which data is input to the processor 1086. The user interface system 1030 also can include a display device, a processor-accessible memory, or any device or combination of devices to which data is output by the processor 1086. The user interface system 1030 and the data storage system 1040 can share a processor-accessible memory.

In various aspects, processor 1086 includes or is connected to communication interface 1015 that is coupled via network link 1016 (shown in phantom) to network 1050. For example, communication interface 1015 can include an integrated services digital network (ISDN) terminal adapter or a modem to communicate data via a telephone line; a network interface to communicate data via a local-area network (LAN), e.g., an Ethernet LAN, or wide-area network (WAN); or a radio to communicate data via a wireless link, e.g., WiFi or GSM. Communication interface 1015 sends and receives electrical, electromagnetic or optical signals that carry digital or analog data streams representing various types of information across network link 1016 to network 1050. Network link 1016 can be connected to network 1050 via a switch, gateway, hub, router, or other networking device.

Processor 1086 can send messages and receive data, including program code, through network 1050, network link 1016 and communication interface 1015. For example, a server can store requested code for an application program (e.g., a JAVA applet) on a tangible non-volatile computer-readable storage medium to which it is connected. The server can retrieve the code from the medium and transmit it through network 1050 to communication interface 1015. The received code can be executed by processor 1086 as it is received, or stored in data storage system 1040 for later execution.

Data storage system 1040 can include or be communicatively connected with one or more processor-accessible memories configured to store information. The memories can be, e.g., within a chassis or as parts of a distributed system. The phrase “processor-accessible memory” is intended to include any data storage device to or from which processor 1086 can transfer data (using appropriate components of peripheral system 1020), whether volatile or nonvolatile; removable or fixed; electronic, magnetic, optical, chemical, mechanical, or otherwise. Exemplary processor-accessible memories include but are not limited to: registers, floppy disks, hard disks, tapes, bar codes, Compact Discs, DVDs, read-only memories (ROM), Universal Serial Bus (USB) interface memory device, erasable programmable read-only memories (EPROM, EEPROM, or Flash), remotely accessible hard drives, and random-access memories (RAMs). One of the processor-accessible memories in the data storage system 1040 can be a tangible non-transitory computer-readable storage medium, i.e., a non-transitory device or article of manufacture that participates in storing instructions that can be provided to processor 1086 for execution.

In an example, data storage system 1040 includes code memory 1041, e.g., a RAM, and disk 1043, e.g., a tangible computer-readable rotational storage device such as a hard drive. Computer program instructions are read into code memory 1041 from disk 1043. Processor 1086 then executes one or more sequences of the computer program instructions loaded into code memory 1041, as a result performing process steps described herein. In this way, processor 1086 carries out a computer implemented process. For example, steps of methods described herein, blocks of the flowchart illustrations or block diagrams herein, and combinations of those, can be implemented by computer program instructions. Code memory 1041 can also store data, or can store only code.

Various aspects described herein may be embodied as systems or methods. Accordingly, various aspects herein may take the form of an entirely hardware aspect, an entirely software aspect (including firmware, resident software, micro-code, etc.), or an aspect combining software and hardware aspects. These aspects can all generally be referred to herein as a “service,” “circuit,” “circuitry,” “module,” or “system.”

Furthermore, various aspects herein may be embodied as computer program products including computer readable program code stored on a tangible non-transitory computer readable medium. Such a medium can be manufactured as is conventional for such articles, e.g., by pressing a CD-ROM. The program code includes computer program instructions that can be loaded into processor 1086 (and possibly also other processors) to cause functions, acts, or operational steps of various aspects herein to be performed by the processor 1086 (or other processor). Computer program code for carrying out operations for various aspects described herein may be written in any combination of one or more programming language(s), and can be loaded from disk 1043 into code memory 1041 for execution. The program code may execute, e.g., entirely on processor 1086, partly on processor 1086 and partly on a remote computer connected to network 1050, or entirely on the remote computer.

### INCORPORATION BY REFERENCE

References and citations to other documents, such as patents, patent applications, patent publications, journals, books, papers, web contents, have been made throughout this disclosure, including to the Supplementary. The Supplementary, and all other such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein.

What is claimed is:

1. A method for measuring propagation velocity from multi-dimensional cardiac imaging, the method comprising:
receiving cardiac imaging data;
estimating local and instantaneous flow propagation velocity (Vₚᵣₒₚ) from the cardiac imaging data; and
employing the local and instantaneous flow propagation velocity to evaluate cardiac flow propagation.

2. The method of claim 1, wherein the cardiac imaging data is 4D magnetic resonance imaging (MRI) data.

3. The method of claim 1, wherein the local and instantaneous flow propagation velocity (Vₚᵣₒₚ) is determined by fitting a first order wave equation to velocity gradients with weighted least-squares.

4. The method of claim 3, wherein the Vp, is estimated from velocity gradients numerically calculated from the velocity fields using second order central (SOC) difference scheme.

5. The method of claim 4, wherein for each timeframe, the Vₚᵣₒₚ at each spatial point is determined by the weighted least-squares fitting of wave propagation equation as:
$${V}_{prop} = arg min ( {\sum }_{i}^{n} {w}_{i}^{2} {\vert \frac{\partial \vec{u}}{\partial t} + {V}_{prop} \cdot ∇ \vec{u} \vert}_{i}^{2} )$$
where n is the total number of data points within the field, and wᵢ is the weight for the i-th data point.

6. The method of claim 5, wherein the i-th data point, is generated based on its spatial distance |Δ [symbol] | from the point of interest as:
$${w}_{i} = \{ \begin{matrix}exp ( - \frac{{\vert Δ \vec{x} \vert}^{2}}{{L}_{0}} & if \vert Δ \vec{x} \vert < {L}_{0} \\ 0 & else\end{matrix}$$
where L₀=0.5 cm is the length scale, yielding a kernel width of 1 cm which corresponds approximately to the radius of the mitral valve.

7. The method of claim 6, wherein weight decreases with increase of the distance |Δ [symbol] |, and only data within L₀ is employed for the fitting.

8. The method of claim 7, wherein the Vₚᵣₒₚ that is dependent on a local flow structure.

9. The method of claim 8, wherein the method further comprising quantifying relative strength of the propagation in a manner in which the Vₚᵣₒₚ component along a direction from mitral orifice towards an apex is extracted and spatially integrated in the LV.

10. The method of claim 9, wherein an integral at each timeframe is normalized by an average of all the timeframes during diastole and is named as propagation intensity (Iₚᵣₒₚ).

11. A system for measuring propagation velocity from multi-dimensional cardiac imaging, the system comprising a processor configured to:
receive cardiac imaging data;
estimate local and instantaneous flow propagation velocity (Vₚᵣₒₚ) from the cardiac imaging data; and
employ the local and instantaneous flow propagation velocity to evaluate cardiac flow propagation.

12. The system of claim 11, wherein the cardiac imaging data is 4D magnetic resonance imaging (MRI) data.

13. The system of claim 11, wherein the local and instantaneous flow propagation velocity (Vₚᵣₒₚ) is determined by fitting a first order wave equation to velocity gradients with weighted least-squares.

14. The system of claim 13, wherein the Vₚᵣₒₚ is estimated from velocity gradients numerically calculated from the velocity fields using second order central (SOC) difference scheme.

15. The system of claim 14, wherein for each timeframe, the Vₚᵣₒₚ at each spatial point is determined by the weighted least-squares (WLS) fitting of a wave propagation equation as:
$${V}_{prop} = arg min ( {\sum }_{i}^{n} {w}_{i}^{2} {\vert \frac{\partial \vec{u}}{\partial t} + {V}_{prop} \cdot ∇ \vec{u} \vert}_{i}^{2} )$$
where n is the total number of data points within the field, and wᵢ is the weight for the i-th data point.

16. The system of claim 15, wherein the i-th data point, is generated based on its spatial distance |Δ [symbol] | from the point of interest as:
$${w}_{i} = \{ \begin{matrix}exp ( - \frac{{\vert Δ \vec{x} \vert}^{2}}{{L}_{0}} & if \vert Δ \vec{x} \vert < {L}_{0} \\ 0 & else\end{matrix}$$
where L₀=0.5 cm is the length scale, yielding a kernel width of 1 cm which corresponds approximately to the radius of the mitral valve.

17. The system of claim 16, wherein weight decreases with increase of the distance |Δ [symbol] |, and only data within L₀ is employed for the fitting.

18. The system of claim 17, wherein the Vₚᵣₒₚ that is dependent on a local flow structure.

19. The system of claim 18, wherein the the processor is further configured to quantify relative strength of the propagation in a manner in which the Vₚᵣₒₚ component along a direction from mitral orifice towards an apex is extracted and spatially integrated in the LV.

20. The system of claim 19, wherein an integral at each timeframe is normalized by an average of all the timeframes during diastole and is named as propagation intensity (Iₚᵣₒₚ).
